(19) United States
(12) Patent Application Publication
(10) Pub. No.: US 2008/0208148 A1
(43) Pub. Date: Aug. 28, 2008

(54) Ophthalmic Device

(75) Inventors: Choon Sen Soon, Singapore (SG); Kee Hong Song, Singapore (SG); Tee Woh Ngiam, Singapore (SG); Bee Suat Lim, Singapore (SG)

Correspondence Address: INTELLECTUAL PROPERTY / TECHNOLOGY LAW, PO BOX 14329, RESEARCH TRIANGLE PARK, NC, 27709, US

(73) Assignee: RENEWE PTE LTD, Singapore (SG)

(21) Appl. No.: 11/915,149

(22) PCT Filed: May 17, 2006
(86) PCT No.: PCT/SG2006/000127

§ 371 Date: Nov. 20, 2007

Related U.S. Application Data
(60) Provisional application No. 60/684,474, filed on May 25, 2005.

(51) Int. Cl. A61H 35/02; A61B 19/02

(52) U.S. Cl. 604/301; 206/438

(57) ABSTRACT

An ophthalmic device for aiding in the accurate dispensing of a medicating substance into an eye, said device comprising an elastic eye cup (4) having an open end and an aperture (8) at an opposite end and a jacket (6) for the eye cup, said jacket being of higher rigidity. The periphery of the open end is adapted to receive and secure the nozzle associated with an eye drop dispenser. The jacket one having a substantially similar shape to the eye cup and having a through-hole sufficient to allow for the secure fitting of said eye cup jacket around a substantial portion of the eye cup, such that the aperture remains unobstructed.

### FIELD OF THE INVENTION

The present invention relates to an ophthalmic device, more specifically, to an ophthalmic device for aiding in the accurate dispensing of a medicating substance into an eye.

### DESCRIPTION OF THE PRIOR ART

The eyes are known to be one of the most sensitive and delicate organs of the human body. The benefit of sight that they confer upon us is extremely important especially in the age where visualization via multimedia means encompasses many areas of our lives. Accordingly, the maintenance of the health of our eyes is of paramount importance to many.

In this regard, one common, albeit less efficient, manner that is presently used in eye care is the utilization of an eye bath. In this method of delivering a medicating substance to an eye, the eye, or least part of it, is immersed into said eye bath. During the immersion, the eye is opened and closed to provide for the distribution of the medicating substance over the surface of the eye. This method requires an amount of freedom of movement of the head of a subject. In addition, the dosage being applied to the eye is not effectively controlled, as the application is rather random in nature.

Apart from the above-mentioned eye bath system, it has been a common practice for subjects to attempt to deliver a medicating substance to the eye directly from an eye drop container. In this instance, the eye drops are frequently dispensed upon the eyelid rather than upon the eye. Once again, there is much wastage of medication incurred by the subject. A more severe consequence of the mentioned problem is that the subject in question does not receive the required medication required for an illness.

Accordingly, there is a need for an ophthalmic device that aids in the efficient delivery of a medicating substance to an eye of a subject. Several devices attempt to overcome the above-mentioned difficulty by the adoption of various strategies. Exemplary representations of the present state of the art are described below.

European patent application EP 0347084 A1 describes an ophthalmic device in the form of a single rigid cup that provides two ports and an eye access. The eye access allows at least a portion of the eye to be covered by the device. The first port is utilized for attaching an eye drop container. The second port is essentially an aperture through which an eye, being subjected to treatment via the device, may view a distinct target area illuminated from outside the device. Accordingly, said device has at least three openings to accommodate the eye drop container, the target aperture and the eye access opening.

Canadian patent application CA 2,027,632 A1 also discloses a rigid one-piece cup having a contoured base adapted to fit closely to the orbital rim of a human eye socket. The device described herein also includes an opening for receiving an eye drop container, with the said opening being at least about 30 mm above the contoured base. The aperture of the said device includes a funnel extending downwards from the said aperture, in order to receive the eye drop container. In an alternative embodiment the aperture may include a pair of arms protruding upwards from the periphery of the aperture to also serve in receiving and securing the eye drop container.

Another example of an ophthalmic device is disclosed in United States patent application US 2005/0043692 A1. This patent application discloses an eye drop holder fabricated entirely as a single piece from a semi-rigid material. The eye drop holder includes an open ended uniform circular base which contacts the region surrounding the eye of a user during operation.

In addition to the above-mentioned patent applications, U.S. Pat. No. 4,733,802 describes a single piece rigid cup having an open end, for access to the eye, and an aperture at an opposite end for receiving and securing an eye drop container. In addition, the said cup includes a flange along the periphery of the eye access opening for supporting the cup against the face of subject. At an opposite end of the flange, a semi-circular opening extending from the periphery of the open end allows the subject to insert one finger to aid in holding the eye-lid open for receiving the dispensed medication.

Although the above-mentioned documents attempt to provide a suitable device for aiding in the dispensation of an eye drop, there still exists a need for an eye drop dispensing system that is able to comfortably fit to the eye socket of a subject. In addition, there exists a need to ensure that said eye drop aid device is mechanically stable to provide sufficient support to an eye drop dispenser container and is cost-effective as well.

### SUMMARY OF THE INVENTION

The present device, having the features defined in the appended independent claim, overcomes the above-mentioned difficulties that exist.

Accordingly, the present invention provides an ophthalmic device for aiding in the accurate dispensing of a medicating substance into an eye. Said device includes an elastic eye cup having an open end and an aperture at an end opposite to the open end, wherein the periphery of the open end is adapted to closely fit the orbital rim of a human eye socket, and the aperture is adapted to receive and secure the nozzle associated with an eye drop dispenser. The device further includes a jacket for the eye cup of higher rigidity, having a substantially complementary shape to the eye cup and having a through-hole sufficient to allow for the secure fitting of said eye cup jacket around a substantial portion of the eye cup, such that the aperture remains unobstructed.

Having a two-piece ophthalmic device, as defined above, with the jacket of higher rigidity allows support of the elastic eyecup and provides a means to grip the elastic eyecup, thereby ensuring that the eyecup can be held safely in place by a user during said use. At the same time, the elastic eyecup also provides a comfortable fit even if pressure is exerted upon it. Accordingly, the present invention provides a simple yet efficient, elegant and aesthetically pleasing solution to the above-mentioned need for a comfortable, mechanically stable eye drop dispensing aid.

### DETAILED DESCRIPTION OF THE INVENTION

In one embodiment, the ophthalmic device for aiding in the accurate dispensing of a medicating substance into an eye is provided. The device has an elastic eyecup and a jacket for the eyecup, wherein the jacket being of higher rigidity. The elastic eyecup includes two ends. The first end is an open end and opposite said first end, the second end has, as an opening, an aperture. The periphery of the open end of the elastic eyecup is adapted to closely fit the orbital rim of a human eye socket. The aperture of the second end is adapted to receive and secure the nozzle and dispensing head associated with an eye drop dispenser. On the surface of the eyecup, there are protrusions. In an exemplary embodiment, there may be two or more of such protrusions. The purpose of the protrusions is to provide anchor points for the rigid jacket to attach.

In one typical embodiment, the jacket of the eyecup is of a substantially complementary shape to the eyecup. In addition, the jacket includes a through-hole, wherein said through-hole is sufficient to allow for the secure fitting of said eye cup jacket around a substantial portion of the eye cup. The through-hole of the jacket is such that it ensures that the aperture remains unobstructed i.e., accessible to receive a dispensing container. Apart from a secure fitting, the through-hole of the jacket may be adapted to cover a substantial portion of the plane of the eyecup in which the aperture is situated. In doing so, the through-hole of the jacket is adapted to accommodate the aperture of the eyecup such that the aperture is capable of receiving a bottle containing eye medicament. In this context, it is noted that the proportion of the eyecup covered by the jacket, regardless of whether the surface of the eyecup that contains the aperture or any other surface is covered by the jacket, may vary.

In addition, the jacket includes undercuts for attaching to the protrusions found on the eyecup. The undercuts and corresponding protrusions allow the jacket to attach to the eyecup via, snap-fit means, double-shot injection means or insert molding means, for example.

A further alternative to securing the jacket to the eyecup may be via adhesive bonding means. In this regard, adhesive bonding refers to the application of a suitable adhesive means, such as glue (‘super glue’), adhesive tape, silicon bonding, or any other common means for bonding for example, between the inner surface of the jacket and the walls of the eyecup.

In a further embodiment, the internal surface of the rigid jacket may have a soft padding, which deforms when the eyecup entered the through-hole of the rigid jacket during assembly of the ophthalmic device. The padding allows for a tight-fit between the eyecup and the rigid jacket.

In one embodiment, the jacket may cover a substantial portion of the eyecup, i.e., for example, up to about 70%-about 90% of the entire external surface area (while maintaining the accessibility of the aperture of the elastic eyecup) in order to provide a strong rigid surface for the subject to grip during use and to improve wear resistance of the elastic eyecup of the device. In addition, the rigid jacket may also provide support to the elastic cup in order to allow the elastic cup to retain its form even when subjected to external forces during the medication application procedure. Alternatively, the jacket may only cover a minor portion of the eyecup, for example, up to about 20%-about 69% of the entire external surface area of the cup.

In another embodiment, the above-mentioned device includes an aperture in the shape of an asterisk, i.e. the aperture is formed from at least six openings, linear in nature. The six openings are symmetrical to each other and originate from a common point of intersection. In other words, if the common point of intersection is taken as the origin, the six symmetrical openings are radial in nature with respect to said origin. Alternatively, the aperture may be formed by four openings or even eight or more openings in a manner similar to that as described above, i.e. said openings (be it four, eight or more) originate in a radial fashion from a point of origin.

Further embodiments of the device may include an aperture in the shape of a circle, a square, an oval or a parallelogram. In principle, any kind of aperture may be used as long as it is sufficient to receive and securely fit the dispensing head of an eye drop container.

In another embodiment, the aperture is positioned such that the origin (centre) of said aperture and the centre of said open end are co-axial. The co-axial arrangement of the aperture and the open end facilitates in the dispensation of the medication into the eye. In this embodiment, due to the co-axial arrangement, the eye-drop container is situated directly above the eye. In an optimal positioning of the device with respect to the eye, the nozzle of the eye drop container should be aligned with that of the pupil, provided the subject looks straight-ahead when the head is in a horizontal (or the body is in a lying down) position.

Alternatively, the aperture may be partially offset from the central axis of the open end. Such an arrangement may remove the requirement for the eye drop container to be in a vertical position when engaged by the aperture. In this embodiment, the eye drop container may be inclined at an angle in order to direct the medication away from the central region (taken to be the pupil of the eye) of the eye, i.e. more towards the cornea. Accordingly, the aperture may be adapted to provide for said inclined engagement of the eye-drop container.

In any of the preceding embodiments, and those that follow, the elastic eyecup may be fabricated from a thermoplastic or elastomers. Examples of thermoplastics and elastomers that may confer such an elastic property and are therefore suitable may be selected from the group consisting of, but not limited to, ethylene-propylene copolymer blends, natural rubber (isopropene), nitrile rubbers and silicone polymers. In this regard, it should be however noted that the class of fabrication materials is by no means limited to the aforesaid but rather, any material that provides the required properties (that it returns to its original shape after undergoing deformation) for the elastic eyecup may be used.

As above, the jacket for the eyecup in any of the preceding embodiments, and those that follow, may be fabricated from a thermoplastic or hard rubber. Examples of thermoplastics that may be used for the fabrication of the jacket include, but are not limited to, ultra-high molecular weight polyethylene (UHMWPE), polypropylene and polyvinylchloride (PVC). However, the fabrication materials are by no means limited to the mentioned material classes but rather, are typically chosen dependent on the property that said material confers to the jacket, i.e. rigidity, aesthetic appearance, degree of adhesiveness to the material of the elastic eyecup and ease of maintenance. In all the embodiments disclosed herein, the jacket should be rigid such that it does not deform or may deform but not substantially enough to vary the existing shape of the eye cup greatly when subjected to an external force (from the grip of a user, for example). In other words, the jacket functions to make rigid the elastic cup to which it is secured thereto.

In another embodiment, the periphery of the open end adapted to closely fit the orbital rim of a human eye socket may include a lining (or padding) of a compressible material. In such an embodiment, the provision of a lining may be to provide additional comfort to the facial region of a user in contact with the eyecup. The compressible material used may be, but is not limited to, rubber, foam, sponge, or any other material that is able to provide an intermediate layer of improved comfort between the eyecup and the facial region surrounding an eye.

In addition to the above, the present device may be used in a kit together with a storage container. The storage container of the invention is adapted to store the ophthalmic device to prevent the accumulation of dust, dirt and bacteria on said device. In this regard, the storage container is of substantially similar shape to the device. Accordingly, it is able to store the device with the jacket being affixed to the eyecup. In alternative embodiments, the interior of the storage container (when sealed with the cover of the container) may be airtight. Such a feature allows a subject to possibly soak the eyecup and jacket in a disinfecting solution in order to maintain a hygienic and bacteria-free ophthalmic device. A further alternative may be to coat the interior of that storage container with a disinfectant that is non-toxic to the eyes of the subject.

The following accompanying drawings, and detailed description of examples of embodiments of the invention, as well as, will further aid in the understanding of the ophthalmic apparatus and its various embodiments according of the present invention. However, said drawings and embodiments should not be construed as limiting the present invention to the illustrated embodiments.

### BRIEF DESCRIPTION OF THE DRAWINGS

FIG. 1A is a perspective view of an ophthalmic device;

FIG. 1B is an isometric view of an eye cup of the ophthalmic device of FIG. 1A;

FIG. 2 is a cross-sectional view of the eye cup about the line A-A;

FIGS. 3A and 3B are top and bottom views of the eye cup respectively;

FIG. 4A is a side view of the length of the eye cup; FIG. 4B is a cross-sectional view of the eye cup about the line B-B;

FIG. 5 is an isometric view of a rigid jacket for the eyecup of the ophthalmic device;

FIG. 6 is a cross-sectional view of the jacket for the eye cup about the line C-C;

FIGS. 7A and 7B are top and bottom views of the rigid jacket respectively;

FIGS. 8A and 8B are perspective views of a storage container for the ophthalmic device from the top and bottom respectively;

FIG. 9A is a bottom view of the storage container; FIG. 9B is a side view of the storage container and FIG. 9C is a top view of the storage container;

FIG. 10A is a cross-sectional view of the storage container from the side and

FIG. 10B is an enlarged view of a selected portion of FIG. 10A; and

FIG. 11 is a cross-sectional view of the ophthalmic device in use.

In the following, examples of the various embodiments of the invention are described including references to the accompanying figures.

### DETAILED DESCRIPTION OF THE EMBODIMENTS

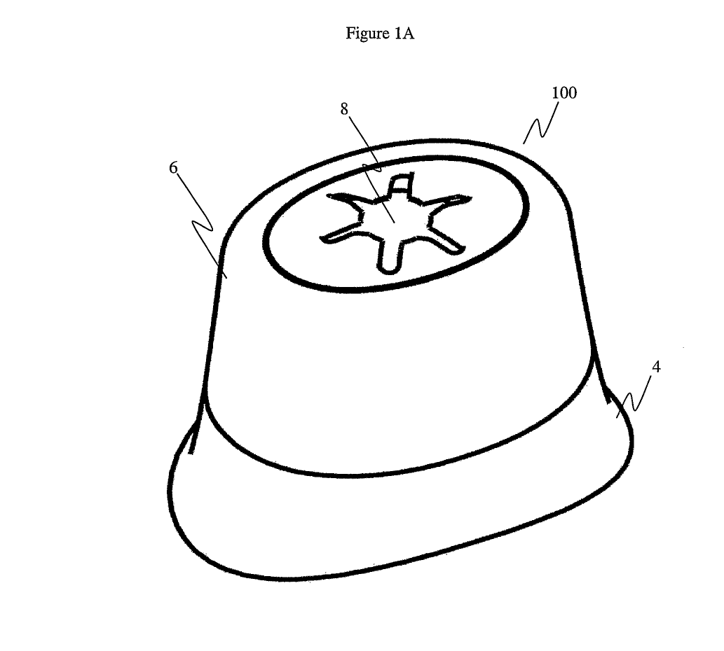

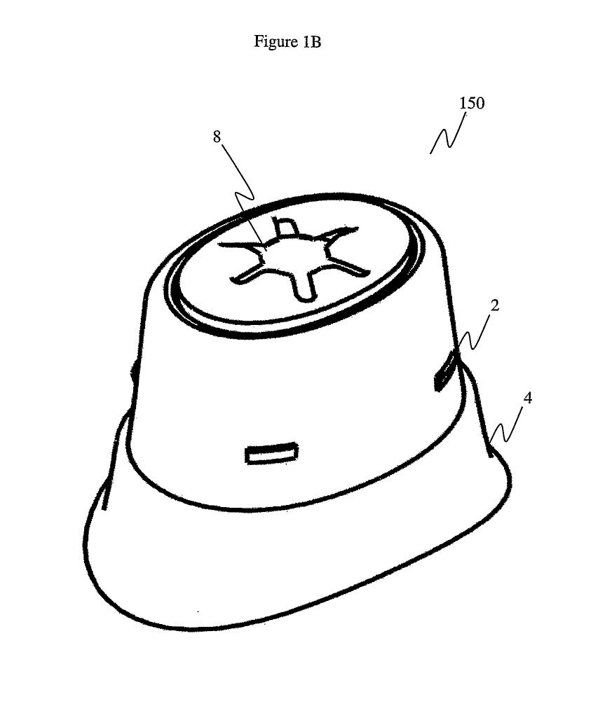

FIG. 1A illustrates an ophthalmic device from a perspective view 100. The ophthalmic device includes an eyecup 4 and a rigid jacket 6, wherein the rigid jacket 6 is attached such that it substantially encompasses the eyecup 4. The eyecup includes an aperture 8, which is also supported by the rigid jacket 6. The rigid jacket 6 is attached to the elastic eyecup 4 by snap-fit means, adhesive bonding means, double-shot injection means, insert-molding means, or any other suitable means, for example.

In a further embodiment, the jacket may be detachable from the eyecup. In such an embodiment, detachable means such as snap-fit and tight fit means may be used instead.

FIG. 1B is a perspective view 150 that illustrates the elastic eyecup 4. The eyecup 4 includes an aperture 8 and protrusion 2. The aperture 8 is in the shape of an asterisk, with six similar linear openings converging to a central point. The central point takes the shape of a dodecagon (12-sided polygon).

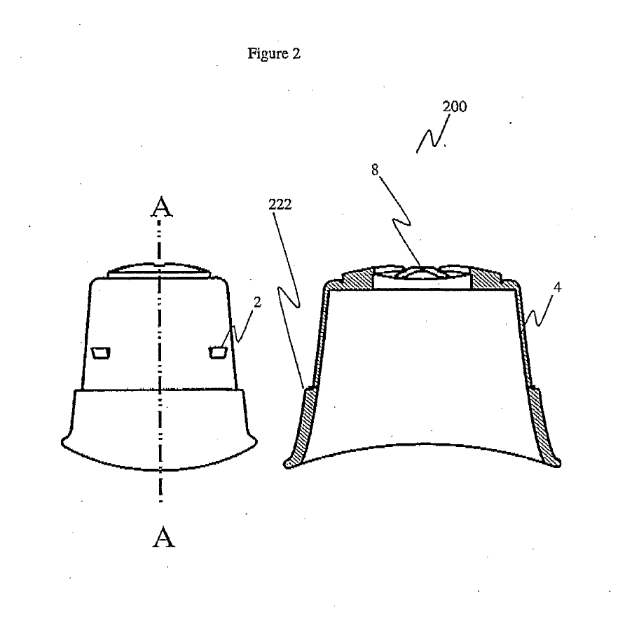

FIG. 2 is a cross-sectional view 200 about the line A-A. The sectional view 200 shows the elastic eyecup 6 detached from the rigid jacket 4. The eyecup 6 includes a step 222 that extends from approximately mid-way of the elastic eyecup 6 to the aperture 8. The step 222 provides sufficient allowance for the rigid jacket 4 to be secured around the elastic eyecup 6. The protrusion 2 acts as an anchoring point for the rigid jacket to the elastic eyecup during any of the above-mentioned attachment mechanisms.

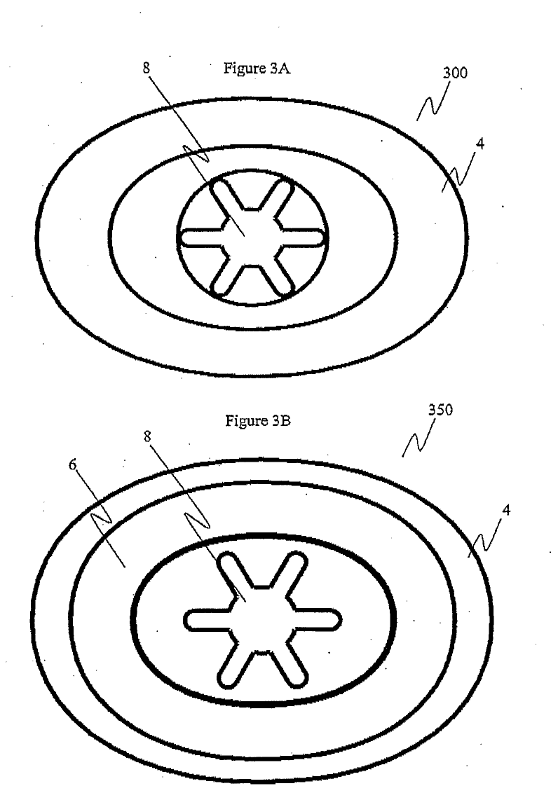

FIG. 3A is a top view 300 of the ophthalmic device showing the aperture 8 of asterisk shape. FIG. 3B is a bottom view 350 of the ophthalmic device also showing the aperture 8 as viewed from within the elastic eyecup 6.

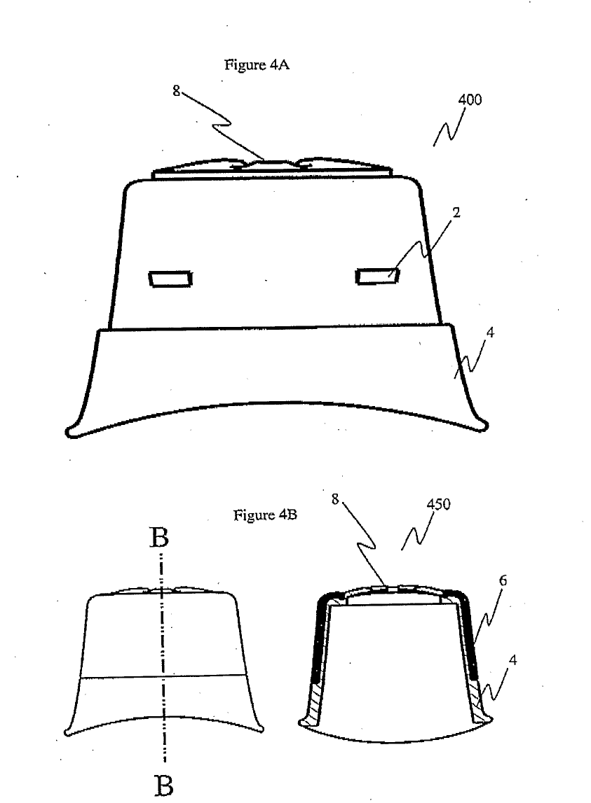

FIG. 4A is a side view 400 of the elastic eyecup 4. The aperture 8 and the protrusion 2 are also shown. FIG. 4B is a cross-sectional view 450 of the eyecup 4 and the rigid jacket 6. The rigid jacket 6 is shown to be supporting a substantial portion of the eyecup 4. The periphery of the aperture 8 is also supported by the rigid jacket 8. This is helpful in order for the aperture 8 to be capable of fitting and securing an eye drop container.

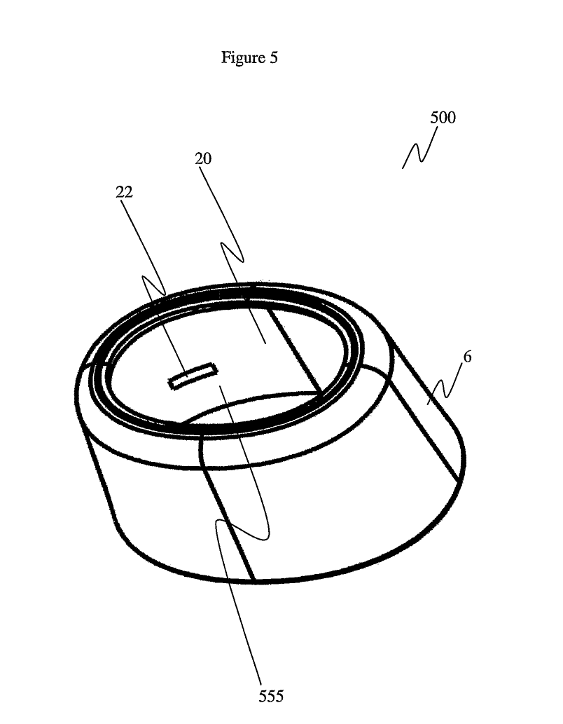

FIG. 5 is a perspective view 500 of the rigid jacket 6. The rigid jacket 6, having a through-hole 555, includes at least two pairs of undercuts 22. The inner surface 20 of the rigid jacket 6 contacts the eyecup 4 in such a manner that said undercuts 22 securely attached to the protrusions 2 of the eyecup 4, through methods such as snap-fit means, adhesive bonding means, double-injection means or insert-molding means, for example. The rigid jacket 6 may also be secured to the elastic eyecup 4 by means of a tight fit when a padded interior wall is present.

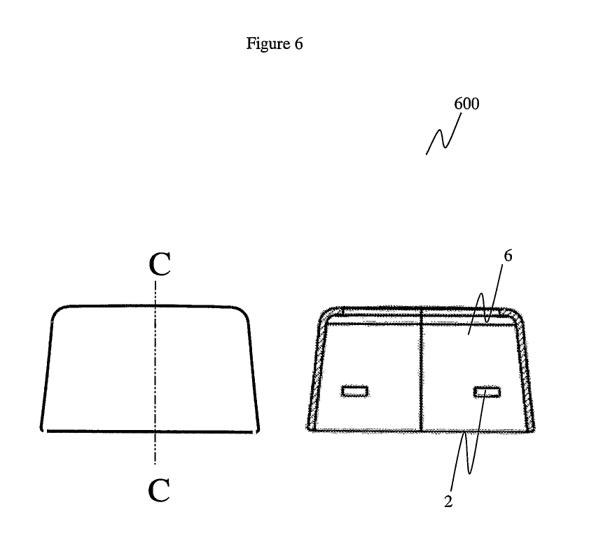

FIG. 6 illustrates a cross-sectional view 600 of the rigid jacket 6 about the line C-C. The sectional view illustrates the openings 22.

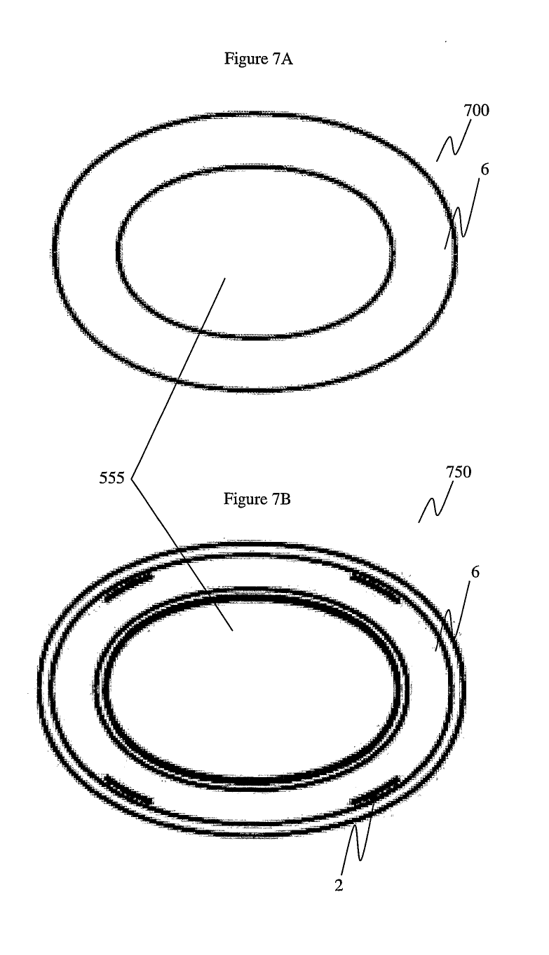

FIG. 7A is a top view 700 of the rigid jacket 6 with the said rigid jacket 6 shown to be having the through-hole 555. FIG. 7B is a bottom view 750 of the rigid jacket 6 also showing the feature of a through-hole along with the openings 22.

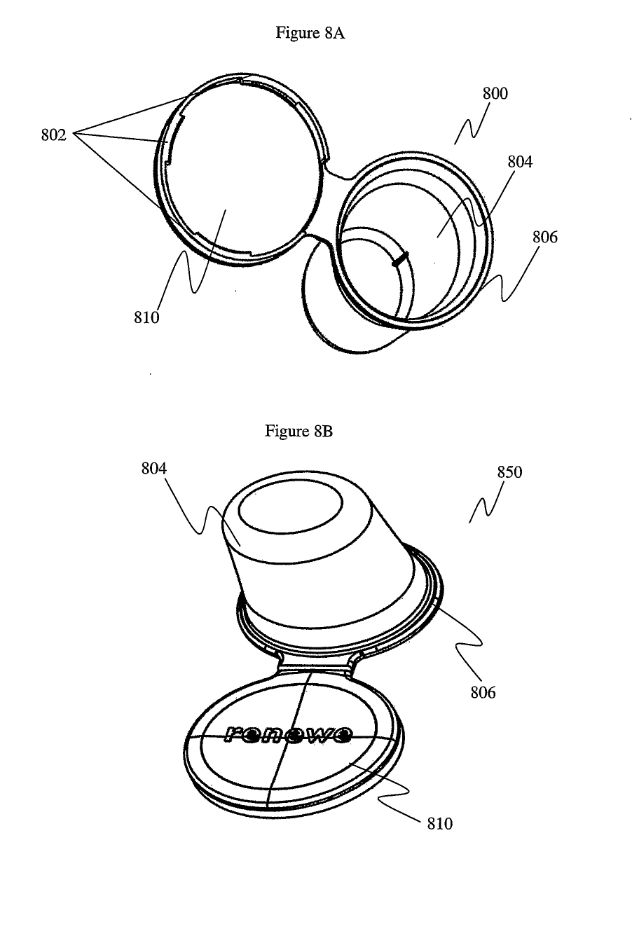

FIG. 8A is a perspective view 800 of a storage container 804. The storage container 804 includes a storage cover 810. Located on the storage cover 810 are three catches 802. The catches 802 are adapted such that the edge 806 will fit into said catch 802 to provide a secure fit and seal for the storage container 804. FIG. 8B is a further perspective view 850 of the bottom of the storage container 804. The perspective view 850 shows the storage cover 810 and the edge 806 to which the storage cover 810 attaches itself to via means of the catches 802.

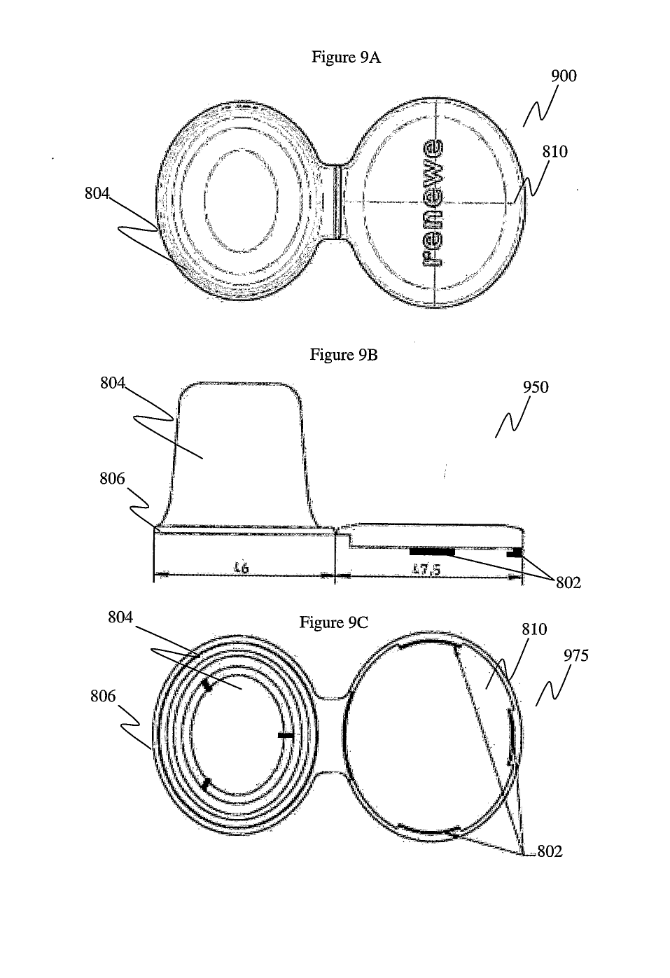

FIG. 9A is a bottom view 900 of the storage container 804, in the open position, for the ophthalmic device. The storage container 804 includes the storage cover 810. FIG. 9B is a side view 950 of the storage container 804. In this illustration, the cover 810 is shown to include a catch 802. The catch 802 is adapted to lock the cover 810 when the storage container 804 is closed. The catch 802 attaches to the edge 806, via snap fit means, when the cover 810 is closed. FIG. 9C is a top view 975 of the storage container 804. The three catches 802 are shown attached to the underside of the cover 810. Said three catches 802 attach to the edge 806 to secure the cover of the storage container 804 when shut.

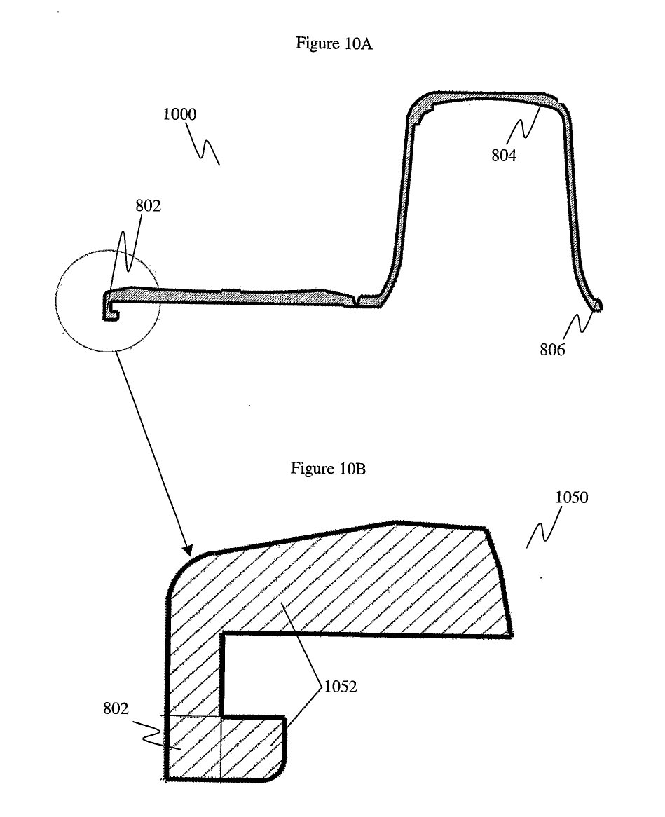

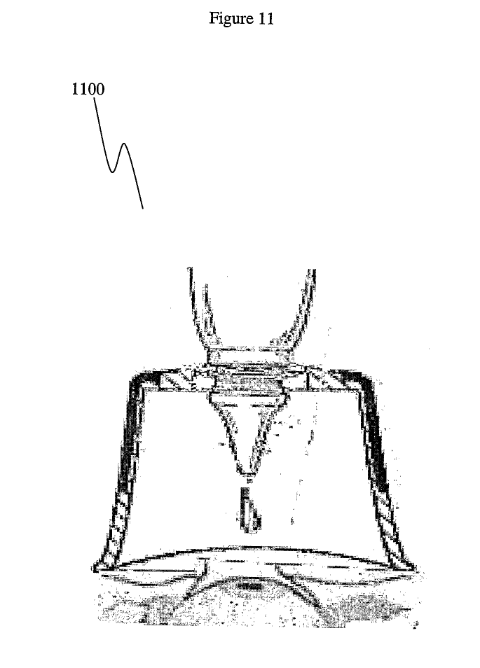

FIG. 10A is another side view 1000 of the storage container 804 wherein the catch 802 and the edge 806 are shown. In this illustration, the catch 802 is enlarged and shown in FIG. 10B, an enlargement 1050. Enlargement 1050 of the catch 802 shows that said catch 802 is of a c-shape with the edge 806 being secured between the two arms 1052 when the cover 810 is closed.

FIG. 11 illustrates the use of the present invention in the application of medication to the eye of a user 1100. The illustration 1100 shows the eye drop container being attached to the ophthalmic device such that said eye drop container is directly over the eye of the user. The aperture of the elastic eyecup grips the neck of the eye drop container to maintain the vertical orientation of the eye drop container thereby ensuring that in the illustrated method of usage, the medication is directed into the central portion of the eye.

The aforementioned description of the various embodiments has been presented for purposes of illustration and description. It is not intended to be exhaustive or to limit the invention to the precise form disclosed, and obviously many modifications and variations are possible in light of the disclosed teaching. It is intended that the scope of the invention be defined by the claims appended hereto.

1. An ophthalmic device for aiding in the accurate dispensing of a medicating substance into an eye, said device comprising:
an elastic eye cup having an open end and an aperture at an end opposite to the open end, wherein the periphery of the open end is adapted to closely fit the orbital rim of a human eye socket, and the aperture is adapted to receive and secure the nozzle associated with an eye drop dispenser, and
a jacket for the eye cup of higher rigidity, having a substantially complementary shape to the eye cup and having a through-hole sufficient to allow for the secure fitting of said eye cup jacket around a substantial portion of the eye cup, such that the aperture of the elastic eyecup remains unobstructed.

2. The ophthalmic device according to claim 1, wherein the elastic eye cup, having at least one protrusion, and the rigid jacket, having at least one opening, are assembled by means of a snap-fit method, an adhesive bonding method, a double-shot injection method or an insert molding method such that the protrusion enters the opening.

3. The device according to claim 1, wherein the aperture is in the shape of an asterisk.

4. The device according to claim 1, wherein the aperture is positioned such that the centre of said aperture and the centre of said open end are co-axial.

5. The device according to claim 1, wherein the elastic eyecup is fabricated from a thermoplastic or rubber.

6. The device according to claim 4, wherein the thermoplastic is selected from a group consisting of an ethylene-propylene copolymer blend, natural rubber (isopropene), nitrile rubber and composites and mixtures thereof.

7. The device according to claim 1, wherein the jacket from the eyecup is fabricated from a thermoplastic or hard rubber.

8. The device according to claim 6, wherein the thermoplastic is selected from the group consisting of ultra-high molecular weight polyethylene (UHMWPE), polypropylene and polyvinylchloride (PVC).

9. The device according to claim 1, wherein periphery of the open end adapted to closely fit the orbital rim of a human eye socket.

10. The device according to claim 1 comprising a lining (or padding) of a compressible material between the orbital rim of the human eye socket and the open end.

11. The device according to claim 10, wherein the compressible material is selected from the group consisting of rubber, foam and sponge.

12. A storage kit comprising:
the device as defined in claim 1, and
a storage container adapted to store the ophthalmic device.
